(19) United States
(12) Patent Application Publication
(10) Pub. No.: US 2018/0000413 A1
(43) Pub. Date: Jan. 4, 2018

(54) MEASUREMENT APPARATUS AND SENSOR SYSTEM

(71) Applicant: Kyocera Corporation, Kyoto-shi, Kyoto (JP)

(72) Inventors: Yuji MASUDA, Yasu-shi (JP); Hiroyuki MORI, Yokohama-shi (JP)

(73) Assignee: Kyocera Corporation, Kyoto-shi, Kyoto (JP)

(21) Appl. No.: 15/543,032

(22) PCT Filed: Jan. 28, 2016
(86) PCT No.: PCT/JP2016/000445

§ 371 Date: Jul. 12, 2017

(30) Foreign Application Priority Data
Jan. 29, 2015 (JP) 2015-015786
Apr. 24, 2015 (JP) 2015-089533

(51) Int. Cl. A61B 5/00; A61B 5/024

(52) U.S. Cl. CPC A61B 5/681; A61B 5/742; A61B 5/02427; A61B 5/02438; A61B 5/7278

(57) ABSTRACT

A measurement apparatus includes a wearing portion to be worn by a subject and at least one sensor supported by the wearing portion and configured to acquire biological information of the subject while in contact with a measured part of the subject. The sensor contacts the measured part at a predetermined pressure or less while the wearing portion is worn by the subject.

### CROSS-REFERENCE TO RELATED APPLICATIONS

This application claims priority to and the benefit of Japanese Patent Application No. 2015-15786 filed Jan. 29, 2015 and Japanese Patent Application No. 2015-89533 filed Apr. 24, 2015, the entire disclosure of these earlier applications being incorporated herein by reference.

### TECHNICAL FIELD

This disclosure relates to a measurement apparatus for measuring biological information and to a sensor system that includes a measurement apparatus for measuring biological information.

### BACKGROUND

Known measurement apparatuses measure biological information from the wrist or other measured part of a subject (user).

### SUMMARY

A measurement apparatus according to this disclosure includes:

a wearing portion to be worn by a subject; and

at least one sensor unit supported by the wearing portion and configured to acquire biological information of the subject while in contact with a measured part of the subject, such that

the sensor unit contacts the measured part at a predetermined pressure or less while the wearing portion is worn by the subject.

Another measurement apparatus according to this disclosure includes:

a wearing portion to be worn by a subject; and

at least one sensor unit supported by the wearing portion and configured to acquire biological information of the subject while in contact with a measured part of the subject, such that

the sensor unit is supported by an elastic body to be displaceable relative to the wearing portion while the wearing portion is worn by the subject.

A sensor system according to this disclosure includes:

a measurement apparatus comprising a wearing portion to be worn by a subject and a sensor unit supported by the wearing portion and configured to acquire biological information of the subject while in contact with a measured part of the subject, the sensor unit contacting the measured part at a predetermined pressure or less while the wearing portion is worn by the subject; and

a display apparatus configured to display biological information by referring to a sensor signal acquired by the sensor unit.

### BRIEF DESCRIPTION OF THE DRAWINGS

In the accompanying drawings:

FIG. 1 is a side view schematically illustrating the configuration of a measurement apparatus according to Embodiment 1;

FIG. 2 is a cross-sectional diagram schematically illustrating the configuration of the measurement unit in FIG. 1;

FIG. 3 is a side view of a first movable member, illustrating the side in a direction orthogonal to the extending direction of a wearing portion;

FIG. 4 illustrates an example of arrangement of an elastic body in the measurement unit;

FIG. 5 schematically illustrates an example of arrangement of biological sensors in the sensor unit;

FIG. 6 schematically illustrates an example of pulse waves acquired by two biological sensors;

FIG. 7 is a cross-sectional diagram schematically illustrating the configuration of the support in FIG. 1;

FIG. 8 is a functional block diagram schematically illustrating the configuration of the measurement apparatus in FIG. 1;

FIG. 9 illustrates an example of a usage state of the measurement apparatus in FIG. 1;

FIG. 10 schematically illustrates a cross-section of the measurement apparatus in FIG. 1 as worn;

FIG. 11 is a graph of the results of an experiment on the relationship between the contact pressure on the wrist and the PWV at close proximity to the wrist;

FIG. 12 illustrates average blood pressure by age group;

FIG. 13 illustrates a modification to the holding state of the measurement unit by the wearing portion;

FIG. 14 illustrates another modification to the holding state of the measurement unit by the wearing portion;

FIG. 15 illustrates the measurement apparatus as worn, illustrating a modification to the wearing portion;

FIG. 16 is an external perspective view of a measurement apparatus according to Embodiment 2;

FIG. 17 is a top view illustrating the wearing portion of the measurement apparatus in FIG. 16;

FIG. 18 is a top view illustrating the interior of the wearing portion in FIG. 17;

FIGS. 19A and 19B are external views of the sensor unit and the substrate according to Embodiment 2;

FIGS. 20A and 20B are cross-sectional views illustrating operations of the measurement apparatus in FIG. 16;

FIGS. 21A, 21B, and 21C illustrate examples of the elastic body according to Embodiment 2;

FIG. 22 illustrates an example of the elastic body according to Embodiment 2;

FIG. 23 further illustrates an example of the elastic body according to Embodiment 2;

FIG. 24 is a functional block diagram schematically illustrating the configuration of the measurement apparatus in FIG. 16;

FIG. 25 illustrates the measurement apparatus as worn, illustrating a modification to the wearing portion of Embodiment 2; and

FIG. 26 schematically illustrates the configuration of a sensor system according to one of the embodiments of this disclosure.

### DETAILED DESCRIPTION

Embodiments of the disclosure are described in detail below, with reference to the drawings.

The measurement apparatus is worn on the wrist with a strap. If the position of the measurement apparatus shifts during measurement of biological information, the contact state between the measurement apparatus and the measured part and the contact pressure of the measurement apparatus on the measured part may change. Consequently, the conditions under which the measurement apparatus acquires biological information may change, making it difficult for the measurement apparatus to obtain stable measurement accuracy of biological information.

Accounting for these considerations, it would be helpful to provide a measurement apparatus that can improve the measurement accuracy of biological information and a sensor system that includes a measurement apparatus that measures biological information.

### Embodiment 1

FIG. 1 is a side view schematically illustrating the configuration of a measurement apparatus according to Embodiment 1 of the disclosure. The measurement apparatus 100 includes a wearing portion 110, a measurement unit 120, and two supports 130.

The measurement apparatus 100 measures the subject's biological information while the measurement apparatus 100 is worn by the subject. The biological information measured by the measurement apparatus 100 is any biological information that can be measured by the measurement unit 120. In this embodiment, an example of the measurement apparatus 100 is described below as acquiring the subject's pulse wave at two locations to measure the pulse wave velocity (PWV).

Further, in this embodiment, the wearing portion 110 is a straight, elongated band. The biological information is measured, for example, after the subject has wrapped the wearing portion 110 of the measurement apparatus 100 around the wrist. In greater detail, the subject wraps the wearing portion 110 around the wrist so that the measurement unit 120 is in contact with the measured part and then performs measurement of biological information. The measurement apparatus 100 measures the PWV of blood flowing through the ulnar artery or the radial artery at the subject's wrist.

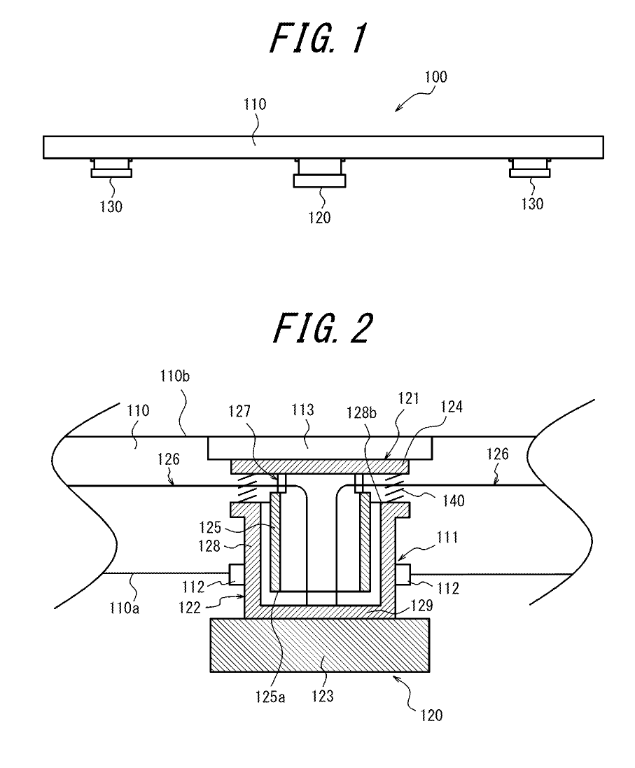

FIG. 2 is a cross-sectional diagram illustrating the configuration of the measurement unit 120 in FIG. 1. Along with the measurement unit 120, FIG. 2 also illustrates the wearing portion 110 in the vicinity of the measurement unit 120.

The wearing portion 110 includes a back face 110a to contact the subject's wrist while worn, and a front face 110b opposite the back face 110a. The wearing portion 110 includes an opening 111 on the back face 110a side. The measurement unit 120 is supported by the wearing portion 110 in a state of protruding from the opening 111 at the back face 110a. Further, the wearing portion 110 includes an elastic member 112 between the opening 111 and the measurement unit 120 to prevent moisture, dust, etc., from entering into the wearing portion 110. The elastic member 112 may, for example, be a flexible waterproof rubber boot. The measurement unit 120 is displaceable within the opening 111 in a direction parallel to the plane of the opening 111.

The wearing portion 110 includes a flat plate member 113 inside the wearing portion 110 at the front face 110b side. The plate member 113 contacts and supports the measurement unit 120 from within the wearing portion 110 at the front face 110b side. In other words, the measurement unit 120 is supported by the wearing portion 110 both by the elastic member 112 at the opening 111 and by the plate member 113. The location of contact between the plate member 113 and the measurement unit 120 is not fixed, and the measurement unit 120 is displaceably supported relative to the wearing portion 110.

The measurement unit 120 includes a first movable member 121, a second movable member 122, and a sensor unit 123.

The first movable member 121 includes a disc-shaped top plate member 124 in contact with the plate member 113 of the wearing portion 110 and a tubular insertion portion 125 that is inserted into the second movable member 122. The outer diameter of the insertion portion 125 is less than the diameter of the top plate member 124. A flexible cable 126 for feeding power to the sensor unit 123 can pass through the space inside the insertion portion 125. Further, on the top plate member 124 side of the insertion portion 125, the first movable member 121 includes cable holes 127 for passage of the flexible cable 126 at both sides in the extending direction of the wearing portion 110.

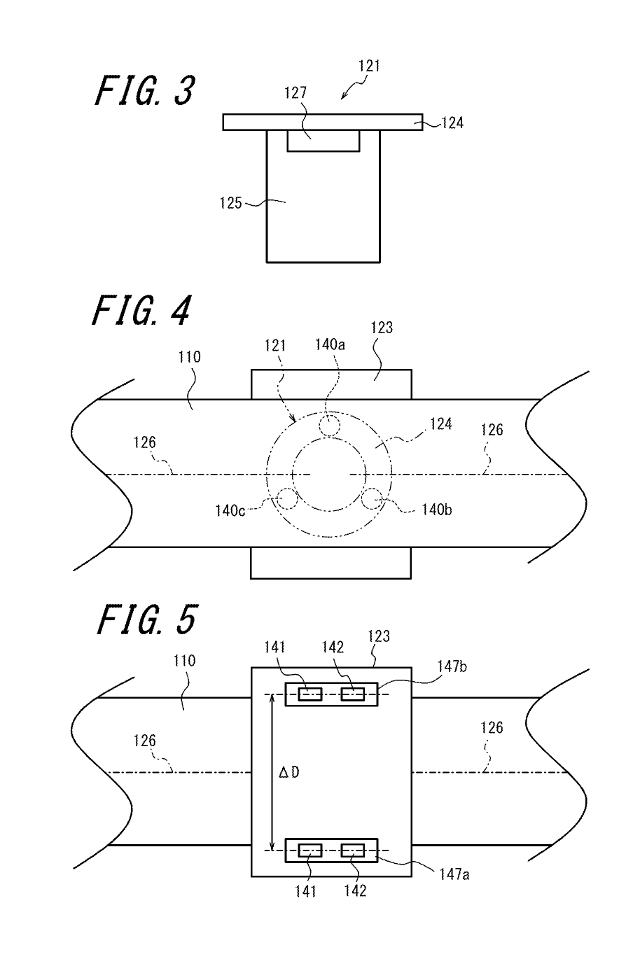

FIG. 3 is a side view of a first movable member 121, illustrating the side in a direction orthogonal to the extending direction of the wearing portion 110. As illustrated in FIG. 3, the first movable member 121 includes the cable holes 127 in the extending direction of the wearing portion 110 as seen from the side.

In FIG. 2, the second movable member 122 includes a receiving portion 128 and a bottom plate 129. The inner diameter of the receiving portion 128 is greater than the outer diameter of the insertion portion 125 of the first movable member 121. In other words, the second movable member 122 is a tube with a bottom. The insertion portion 125 is inserted into the receiving portion 128. At the open end 128b of the receiving portion 128, the second movable member 122 is connected to the top plate member 124 of the first movable member 121 by an elastic body 140 that can expand and contract. When the elastic body 140 is neither expanded nor contracted, the insertion portion 125 and the receiving portion 128 are held along the same axis. In this embodiment, the elastic body 140 is a spring, for example. The elastic body 140 is not limited to being a spring, however, and may be any elastic body.

In this embodiment, the measurement unit 120 includes three elastic bodies 140. The plurality of elastic bodies 140 are positioned so as not to interfere with the flexible cable 126. FIG. 4 illustrates an example of arrangement of the elastic bodies 140 in the measurement unit 120. FIG. 4 illustrates the measurement unit 120 from the upper surface thereof (the top in FIG. 1). In this top view, the position of the first movable member 121 is indicated by dashed double-dotted lines, and the position of the elastic body 140 is indicated by dashed lines.

As illustrated in FIG. 4, the three elastic bodies 140a, 140b, and 140c are disposed at equal intervals along the circumference of the top plate member 124. With this arrangement, the elastic bodies 140 can support the top plate member 124 of the first movable member 121 without interfering with the flexible cable 126 indicated in FIG. 4 by the dashed dotted line. In this embodiment, the case of three elastic bodies 140 has been described, but the number of elastic bodies 140 in the measurement unit 120 is not limited to three. The measurement unit 120 may include any number of elastic bodies 140 at positions that do not interfere with the flexible cable 126.

Referring again to FIG. 2, when the elastic bodies 140 are neither expanded nor contracted, the open end 125a of the insertion portion 125, which has no top plate member 124, and the bottom plate 129 are separated from each other. Also, when the elastic bodies 140 are neither expanded nor contracted, the outer peripheral surface of the insertion portion 125 and the inner peripheral surface of the receiving portion 128 are separated from each other. Thus, because the insertion portion 125 of the first movable member 121 and the second movable member 122 have a gap therebetween, the first movable member 121 and the second movable member 122 are displaceable with respect to each other in the direction of the front face 110b and the back face 110a of the wearing portion 110 (i.e. vertically). Also, the first movable member 121 and the second movable member 122 are also displaceable in a plane parallel to the extending direction of the wearing portion 110. Furthermore, the first movable member 121 and the second movable member 122 are displaceable so that the axes of the insertion portion 125 and the receiving portion 128 shift and tilt.

The sensor unit 123 is connected to the second movable member 122 and is displaced in conjunction with displacement of the second movable member 122. The sensor unit 123 is displaceable with respect to the wearing portion 110 in the direction of the front face 110b and the back face 110a of the wearing portion 110 (i.e. vertically). The sensor unit 123 is displaceable with respect to the wearing portion 110 in a plane parallel to the extending direction of the wearing portion 110. Furthermore, the sensor unit 123 is also displaceable so as to tilt relative to the front face 110b and the back face 110a of the wearing portion 110. The sensor unit 123 includes biological sensors that acquire biological information on the subject. FIG. 5 illustrates an example of arrangement of the biological sensors in the sensor unit 123. FIG. 5 illustrates the sensor unit 123, viewing the wearing portion 110 from the back face 110a side. The dimension of the sensor unit 123 in the width direction of the wearing portion 110 is greater than the width of the wearing portion 110. In other words, as viewed from the upper surface of the measurement apparatus 100 (the top in FIG. 1), the sensor unit 123 projects beyond the wearing portion 110. The sensor unit 123 measures biological information on the subject while in contact with the subject's measured part. As illustrated in FIG. 5, the sensor unit 123 in this embodiment includes two sensors arranged according to a predetermined interval: a biological sensor 147a and a biological sensor 147b. The interval ΔD between the biological sensor 147a and the biological sensor 147b is, for example, 10 mm to 30 mm. The biological sensor 147a and the biological sensor 147b acquire the pulse wave at different measured parts by an optical method. The pulse wave refers to a waveform representation, from the body surface, of the change in volume over time in a blood vessel due to inflow of blood.

The biological sensors 147a and 147b for example are each provided with a pair of an optical emitter 141 and an optical detector 142. The optical emitter 141 emits a measuring beam onto the measured part, the measuring beam passes through the body, and the optical detector 142 acquires the pulse wave by detecting light reaching the optical detector 142. The optical emitter 141 includes a light emitting element such as a Light Emitting Diode (LED) or a Laser Diode (LD). The optical detector includes a light detecting element, such as a Photodiode (PD) or a Phototransistor (PT).

The optical emitter 141 for example emits green light (wavelength: 500 nm to 550 nm), red light (wavelength: 630 nm to 780 nm), or near infrared light (wavelength: 800 nm to 1600 nm). As compared to light of shorter wavelengths, light of longer wavelengths does not diminish until reaching a deeper position within the body. Therefore, by measuring biological information using an element that emits near infrared light, the measurement accuracy can be improved over the case of using an element that emits green light or red light.

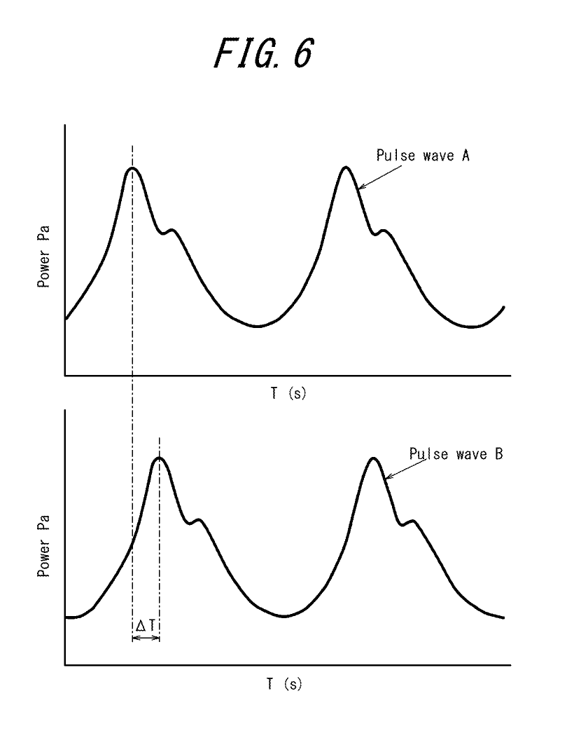

The position of the sensor unit 123 relative to the measured part is adjusted so that the biological sensors 147a and 147b are both above the ulnar artery or the radial artery. Next, the method for measuring the PWV at close proximity to the wrist using two acquired pulse waves is described with reference to FIG. 6.

FIG. 6 illustrates an example of pulse waves acquired by two biological sensors. The sensor unit 123 has been adjusted by the user so that the biological sensors 147a and 147b are both above the radial artery. In FIG. 6, the pulse wave A acquired by the sensor 147a in contact with a first measured part A on the radial artery and the pulse wave B acquired by the sensor 147b in contact with a second measured part B on the radial artery are arranged vertically for comparison. The two acquired pulse waves are synchronized. The PWV (m/s) can be calculated by the following formula, where ΔT (ms) is the interval between the peak times in the two acquired pulse waves, and ΔD (mm) is the distance between the biological sensors 147a and 147b.

PWV=ΔD/ΔT

The flexible cable 126 is connected to the sensor unit 123. The flexible cable 126 for example feeds power to the sensor unit 123 from a power source provided inside the wearing portion 110. The flexible cable 126 for example also supplies a control signal to the sensor unit 123 from a controller provided inside the wearing portion 110. The flexible cable 126 for example supplies the biological information acquired by the sensor unit 123 to the controller provided inside the wearing portion 110.

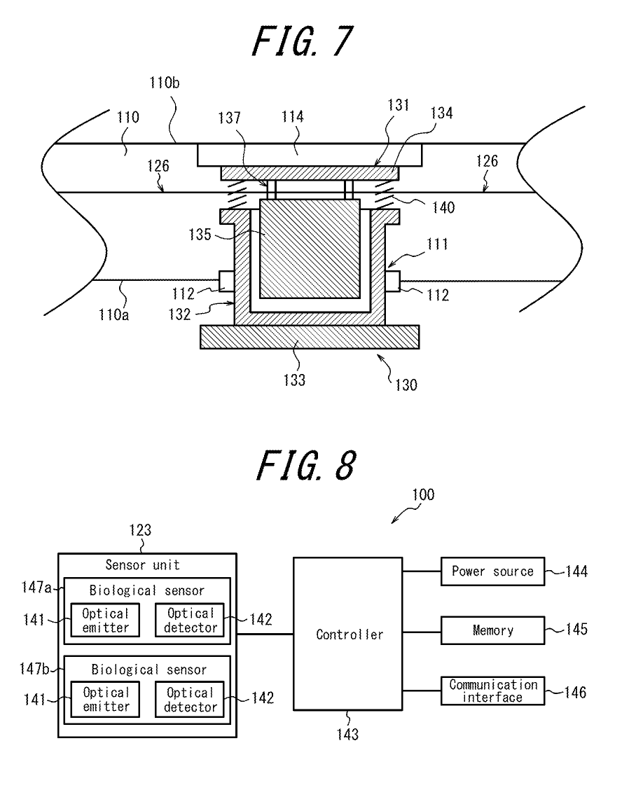

Next, the structure of the supports 130 is described. FIG. 7 is a cross-sectional diagram illustrating the configuration of the supports 130 in FIG. 1. In this embodiment, each support 130 includes a first movable member 131, a second movable member 132, and a support plate 133. The structure of the support 130 is similar to that of the measurement unit 120, in that the first movable member 131 and the second movable member 132 are connected to be displaceable with respect to each other by the elastic bodies 140. The differences in the support 130 from the measurement unit 120 are described below.

In the support 130, the support plate 133 is disposed at the position where the sensor unit 123 is disposed in the measurement unit 120. The support plate 133 is a plate-shaped member that contacts the wrist and supports the measurement apparatus 100 when the subject wraps the measurement apparatus 100 around the wrist. The support plate 133 may be configured as a deformable member that conforms to the curved surface of the contacted wrist when the subject wraps the measurement apparatus 100 around the wrist.

Like the insertion portion 125 of the first movable member 121 in the measurement unit 120, an insertion portion 135 of the first movable member 131 in the support 130 includes cable holes 137 penetrating the first movable member 131 for passage of the flexible cable 126 at the top plate member 134 side. On the other hand, since the support 130 has no sensor unit 123 requiring a power supply, the inside of the insertion portion 135 of the support 130 need not be hollow, unlike the insertion portion 125 of the measurement unit 120.

The dimension of the support 130 in the width direction of the wearing portion 110 at a plate member 114 in contact with the top plate member 134 is less than the width of the wearing portion 110. In other words, as viewed from the upper surface of the measurement apparatus 100 (the top in FIG. 1), the plate member 114 is covered by the wearing portion 110. Like the measurement unit 120, the support 130 is displaceably supported relative to the wearing portion 110.

FIG. 8 is a functional block diagram schematically illustrating the structure of the measurement apparatus 100 in FIG. 1. The measurement apparatus 100 includes the sensor unit 123, a controller 143, a power source 144, a memory 145, and a communication interface 146. In this embodiment, the sensor unit 123 is included in the measurement unit 120, and the controller 143, power source 144, memory 145, and communication interface 146 are included inside the wearing portion 110.

The sensor unit 123 includes the above-described biological sensors 147a and 147b and acquires biological information from the measured parts. Furthermore, the biological sensors 147a and 147b each include the optical emitter 141 and optical detector 142.

The controller 143 is a processor that, starting with the functional blocks of the measurement apparatus 100, controls and manages the measurement apparatus 100 overall. The controller 143 is also a processor that calculates the PWV using the acquired pulse waves. The controller 143 is configured as a processor such as a Central Processing Unit (CPU) that executes a program prescribing control procedures and a program that calculates the PWV. These programs are stored in a storage medium such as the memory 145, for example.

The power source 144 for example includes a lithium-ion battery and a control circuit for charging and discharging the battery. The power source 144 supplies power to the measurement apparatus 100 overall.

The memory 145 may be configured with a semiconductor memory, a magnetic memory, or the like. The memory 145 stores a variety of information, programs for causing the measurement apparatus 100 to operate, and the like and also functions as a working memory. The memory 145 for example may store the measurement result of the sensor unit 123 measuring biological information.

The communication interface 146 exchanges a variety of data with an external apparatus by wired or wireless communication. The communication interface 146 for example communicates with an external apparatus storing biological information of the subject and transmits the measurement results of biological information measured by the measurement apparatus 100 to the external apparatus.

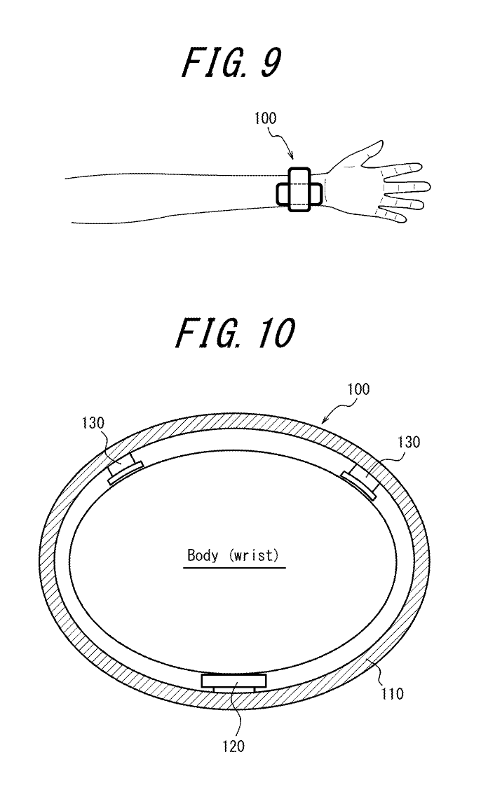

Next, the method for using the measurement apparatus 100 is described. FIG. 9 illustrates an example of a state of usage of the measurement apparatus 100 by the subject. As illustrated in FIG. 9, the subject uses the measurement apparatus 100 by wrapping the measurement apparatus 100 around the wrist. When wrapping the measurement apparatus 100 around the wrist, the subject wraps the wearing portion 110 around the wrist after adjusting the position of the measurement unit 120 so that a measurement beam is emitted from the optical emitter 141 of the sensor unit 123 in the measurement unit 120 onto the ulnar artery or the radial artery for which biological information is to be acquired.

FIG. 10 schematically illustrates a cross-section of the measurement apparatus 100 as worn. The measurement apparatus 100 is worn by the subject in a state such that the measurement unit 120 and the two supports 130 are in contact with the wrist. The measurement unit 120 preferably is made to contact the wrist at a position where the measurement beam is emitted onto the ulnar artery or the radial artery, by adjustment at the time the subject wears the measurement apparatus 100.

While the measurement apparatus 100 is worn, the measurement unit 120 and the two supports 130 are in close contact with the subject's wrist because of the elastic force of the elastic bodies 140. By the measurement unit 120 being in close contact with the wrist, the positional relationship between the wrist and the measurement unit 120 tends not to change, allowing improvement in the measurement accuracy of the measurement unit 120.

As explained with reference to FIGS. 2 and 7, the wearing portion 110 is not fixed to the measurement unit 120 and the supports 130. Rather, the measurement unit 120 and the supports 130 are displaceably supported relative to the wearing portion 110. Therefore, if the wearing portion 110 shifts relative to the wrist, which is the measured part, the positional relationship of the wearing portion 110 relative to the measurement unit 120 and the supports 130 shifts. As a result, the measurement unit 120 and the supports 130, which are in close contact with the wrist, tend not to change position relative to the wrist (and the measured part). When the wearing portion 110 shifts relative to the wrist, the first movable members 121 and 131 and the second movable members 122 and 132 in the measurement unit 120 and the supports 130 are displaced. The close contact of the measurement unit 120 and the supports 130 with the wrist is thereby easily maintained. Therefore, the positional relationship between the measurement unit 120 and the wrist tends to remain unchanged, and the conditions for measurement of biological information by the measurement unit 120 do not change easily.

While the measurement apparatus 100 is worn, the sensor unit 123 of the measurement unit 120 is in contact with the wrist at a predetermined pressure or less. During measurement of biological information, the sensor unit 123 may always contact the wrist at a predetermined pressure or less, regardless of movement by the subject. The predetermined pressure is determined on the basis of factors such as the biological information measured by the measurement apparatus 100 and the configuration of the measurement apparatus 100. The predetermined pressure is preferably a pressure at which error tends not to occur in the measurement results of the biological information. In this embodiment, in order for the measurement apparatus 100 to measure the PWV as the biological information, the predetermined pressure is preferably a pressure at which error tends not to occur in the measurement results of the PWV.

The following describes preferred values for the predetermined pressure in the measurement apparatus 100 according to this embodiment. FIG. 11 is a graph of the results of an experiment on the relationship between the contact pressure on the wrist by the sensor unit 123 of the measurement apparatus 100 and the PWV at close proximity to the wrist, illustrating the results of an experiment performed on a subject with an average blood pressure of approximately 95 mmHg. Here, the average blood pressure indicates the average blood pressure in the arteries and is calculated with the following formula, using the systolic blood pressure (maximum blood pressure) and the diastolic blood pressure (minimum blood pressure).

average blood pressure=((maximum blood pressure)+(minimum blood pressure)×2)÷3

In FIG. 11, when the contact pressure of the sensor unit 123 on the wrist is equivalent to the subject's average blood pressure (approximately 95 mmHg), then the pressure difference between the blood pressure inside the blood vessel and the contact pressure on the blood vessel from the outside is zero on average. Hence, the blood flow is not easily affected by the contact pressure. Therefore, the measurement result of PWV by the measurement apparatus 100 tends to improve.

When the contact pressure is less than the subject's average blood pressure (95 mmHg), the expansion and contraction of the blood vessel wall tends not to be affected by the contact pressure, making the elasticity of the blood vessel wall nearly constant. Nearly constant results are also obtained for measurement of the PWV. When the contact pressure is less than a predetermined pressure (approximately 50 mmHg in FIG. 11), however, the sensor unit 123 and the measured part are not sufficiently in contact. It then becomes difficult to acquire the pulse wave and to measure the PWV.

Conversely, when the contact pressure is higher than the subject's average blood pressure (95 mmHg), the elasticity of the blood vessel wall is affected by the contact pressure and decreases with an increase in the contact pressure. As a result, the PWV also decreases with an increase in the contact pressure.

In other words, the experiment results illustrated in FIG. 11 demonstrate that the measurement accuracy of the PWV is not seriously impaired if the contact pressure is between the contact pressure at which the PWV is measurable (approximately 50 mmHg in FIG. 11) and the subject's average blood pressure (approximately 95 mmHg in FIG. 11). Conversely, if the contact pressure is greater than the subject's average blood pressure, the measurement accuracy tends to decrease. Therefore, the measurement unit 120 preferably contacts the measured part at a predetermined pressure equal to or less than the subject's average blood pressure.

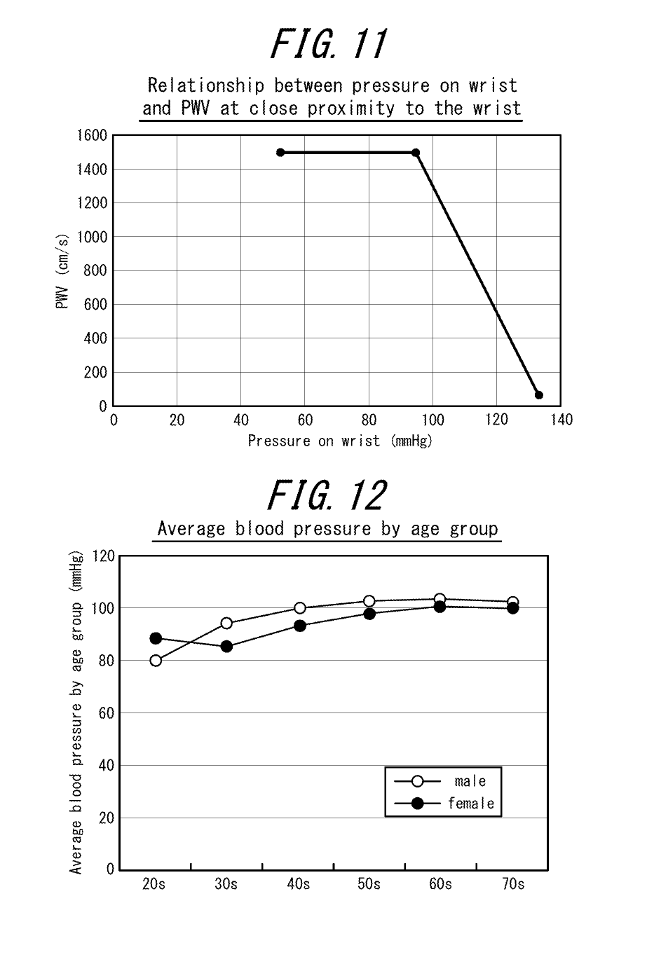

FIG. 12 illustrates the average blood pressure by age group on the basis of the Fifth National Survey of Cardiovascular Diseases published by the Japanese Ministry of Health, Labour, and Welfare. To achieve versatility, the measurement apparatus 100 is for example preferably usable by adults aged 20 and over. As described above, the measurement accuracy is not seriously impaired when the contact pressure is at most the subject's average blood pressure. In light of this, the predetermined pressure in the measurement apparatus 100 in this embodiment is preferably approximately 80 mmHg, which is the average pressure for a 20-year-old male and the lowest average pressure in FIG. 12. In other words, the measurement apparatus 100 is configured so that when worn, the measurement unit 120 contacts the measured part at a pressure of 80 mmHg or less. Elastic bodies that can achieve this pressure are used in the measurement apparatus 100 as the elastic bodies 140.

In this way, with the measurement apparatus 100 according to this embodiment, by virtue of the measurement unit 120 (sensor unit 123) that measures the biological information contacting the measured part at a predetermined pressure or less, the measurement accuracy of biological information can be improved by the setting of the predetermined pressure. For example, in the above embodiment, the measurement accuracy of the PWV can be improved by setting the predetermined pressure to 80 mmHg.

Since the sensor unit 123 is displaceably supported relative to the wearing portion 110, the relative positional relationship between the sensor unit 123 and the wearing portion 110 changes if the wearing portion 110 shifts during measurement of biological information. As a result, the relative positional relationship between the measured part and the sensor unit 123 that is in close contact with the measured part does not change easily. Therefore, with the measurement apparatus 100, the measurement conditions do not change easily with respect to the position of the sensor unit 123 relative to the measured part during measurement of biological information, allowing improvement in the measurement accuracy of biological information. Also, when the subject wears the measurement apparatus 100, the sensor unit 123 and the two supports 130 are in contact with the subject, so that the subject feels less pressure at the points of contact with the measurement apparatus 100, as compared to when the entire wearing portion 110 is in contact with the wrist.

In this embodiment, for example, the holding state of the measurement unit 120 and the supports 130 in the wearing portion 110 is not limited to the example in FIGS. 2 and 7. The wearing portion 110 can hold the measurement unit 120 and the supports 130 with a different appropriate structure. The following describes a modification to the measurement unit 120. Since a modification to the supports 130 is similar to the modification to the measurement unit 120, a description thereof is omitted.

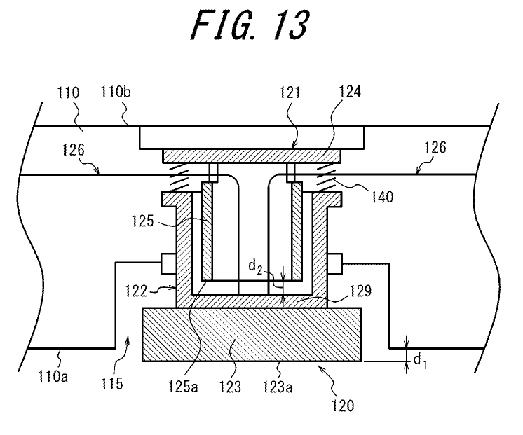

FIG. 13 illustrates a modification to the holding state of the measurement unit 120 by the wearing portion 110 and is a cross-sectional diagram corresponding to FIG. 2 in the above embodiment. In the example illustrated in FIG. 13, the wearing portion 110 includes a recess 115 that contains the measurement unit 120. The portion of the second movable member 122 projecting from the wearing portion 110 at the sensor unit 123 side in the above embodiment is covered by the wearing portion 110 in this modification so as to be positioned within the recess 115. When the elastic bodies 140 are neither expanded nor contracted, a contact surface 123a where the sensor unit 123 contacts the measured part projects from the recess 115 by a distance d1 from the back face 110a of the wearing portion 110. The distance d1 is preferably shorter than the distance d2 between the open end 125a side of the insertion portion 125 and the bottom plate 129. By virtue of the distance d1 being shorter than the distance d2, when the subject is wearing the measurement apparatus 100 on the wrist, the measured part can push up on the contact surface 123a towards the front face 110b until the contact surface 123a is flush with the back face 110a. The elastic bodies 140 are preferably configured so that when the contact surface 123a is flush with the back face 110a, the pressure from the sensor unit 123 on the measured part becomes a predetermined pressure. Since the contact surface 123a is not further displaced towards the front face 110b from being flush with the back face 110a, a pressure that exceeds the predetermined pressure on the measured part, which would reduce the measurement accuracy of the biological information, can easily be prevented when the measurement apparatus 100 is worn. As a result, during measurement of biological information, the sensor unit 123 can always contact the wrist at a predetermined pressure or less, regardless of movement by the subject.

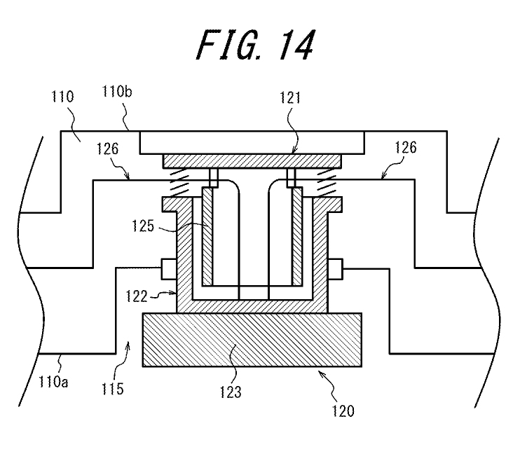

FIG. 14 illustrates another modification to the holding state of the measurement unit 120 by the wearing portion 110 and is a cross-sectional diagram corresponding to FIG. 2 in the above embodiment. In the example illustrated in FIG. 14, the wearing portion 110 includes a recess 115 as in the example in FIG. 13, but at locations other than where the measurement unit 120 is supported in the wearing portion 110, the wearing portion 110 is thinner than in the example in FIG. 13. As a result, the weight of the measurement apparatus 100 can be reduced, reducing the sense of discomfort and the burden on the subject who is wearing the measurement apparatus 100 on the wrist.

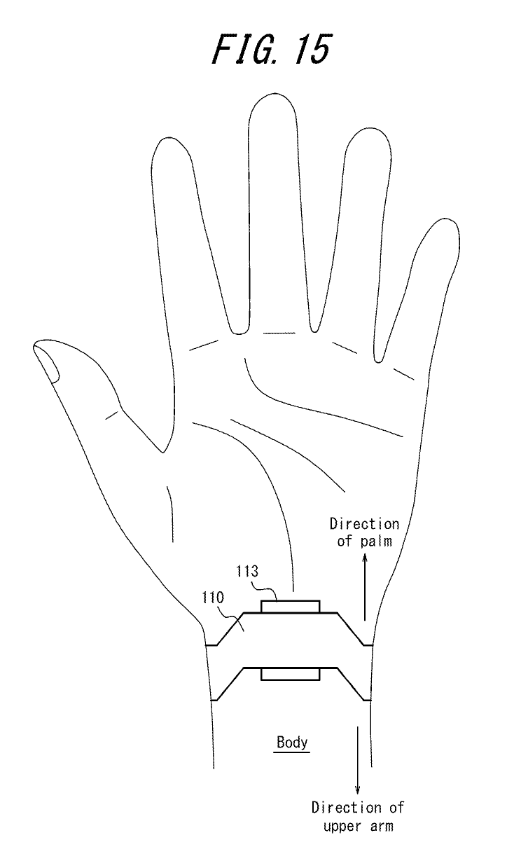

The wearing portion 110 has been described as being straight in the above embodiment, but the wearing portion 110 need not be straight. For example, when the subject is wearing the measurement apparatus 100 on the wrist, at least a portion of the wearing portion 110 may be offset in the direction of the upper arm. In the example in FIG. 15, when the measurement apparatus 100 is worn, the location of the measurement unit 120 in the wearing portion 110 is above the wrist, whereas the remainder is offset in the direction of the upper arm. As a result, the measurement unit 120 is in contact with the measured part of the wrist, whereas the remainder of the wearing portion 110 is positioned towards the upper arm from the wrist. Movement of the subject's wrist is therefore less impeded.

The following describes Embodiment 2 of this disclosure.

### Embodiment 2

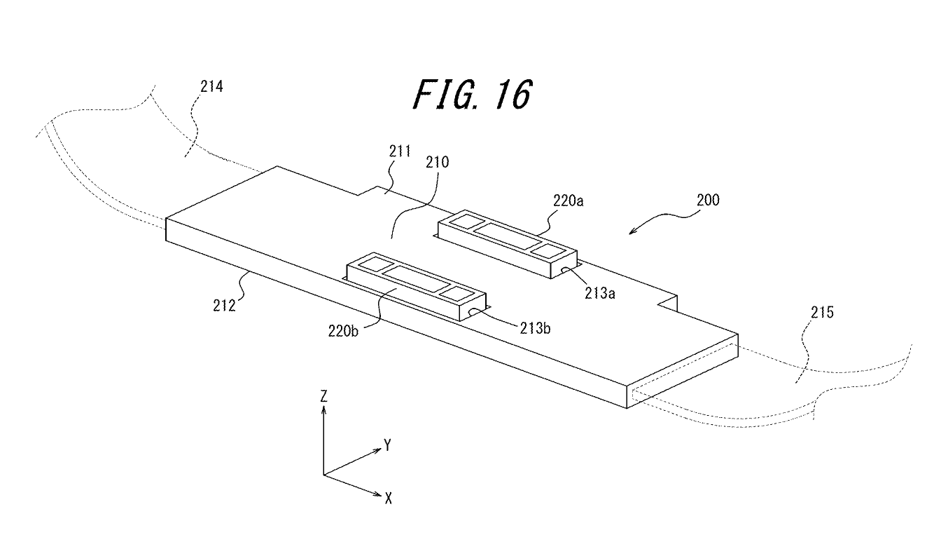

FIG. 16 is a perspective view schematically illustrating the configuration of a measurement apparatus according to Embodiment 2. The measurement apparatus 200 according to Embodiment 2 includes a wearing portion 210 and a plurality of sensor units 220a, 220b. As illustrated in FIG. 16, the wearing portion 210 constituting the housing of the measurement apparatus 200 includes a back face 211 facing the positive direction of the z-axis shown in the diagram and a front face 212 facing the negative direction of the z-axis. The measurement apparatus 200 is used by being worn with the back face 211 of the wearing portion 210 on the measured part of the subject's body. Therefore, when the subject is wearing the wearing portion 210 of the measurement apparatus 200 on the wrist, the subject can view the front face 212 of the wearing portion 210.

The wearing portion 210 of the measurement apparatus 200 includes openings 213a, 213b on the back face 211 side. The first sensor unit 220a projects from the opening 213a, and the second sensor unit 220b projects from the opening 213b.

Since the wearing portion 210 is used while worn by the subject, the wearing portion 210 for example preferably includes members such as bands 214, 215. In FIG. 16, as one example, bands 214, 215 used for wrapping around the subject's arm are partially illustrated by dashed lines. These bands 214, 215 are not limited to the configuration in FIG. 16 and may have any configuration allowing the subject to wear the wearing portion 210. In this way, the wearing portion 210 in this embodiment can be a band worn on the subject's wrist.

The measurement apparatus 200 measures the subject's biological information while the measurement apparatus 200 is worn by the subject. The biological information measured by the measurement apparatus 200 may be any biological information that can be measured using the measurement unit 220. In this embodiment, as one example, the measurement apparatus 200 is described as acquiring the subject's pulse wave at two locations to measure the PWV.

In this embodiment, the wearing portion 210 may be an elongated belt-shaped band. The biological information is measured for example after the subject has wrapped the wearing portion 210 of the measurement apparatus 200 around the wrist. In greater detail, the subject wraps the wearing portion 210 around the wrist so that the plurality of sensor units 220a, 220b are in contact with the measured part and then measures biological information. The measurement apparatus 200 measures the PWV of blood flowing through the ulnar artery or the radial artery at the subject's wrist.

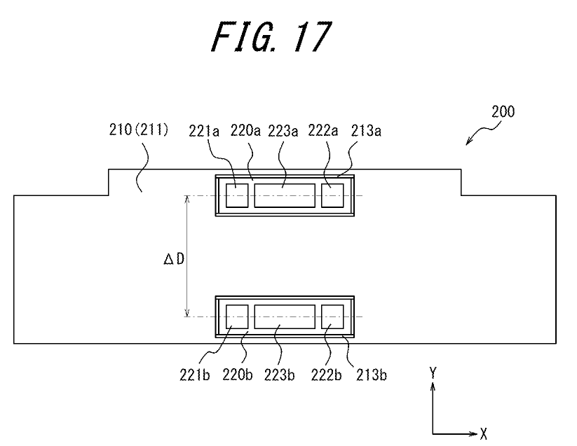

FIG. 17 illustrates the back face 211 of the wearing portion 210 in the measurement apparatus 200 illustrated in FIG. 16. The bands 214, 215 are omitted from the diagrams from FIG. 17 onward.

The plurality of sensor units 220a, 220b each include a biological sensor that acquires biological information on the subject. FIG. 17 illustrates an example of arrangement of the biological sensors in the sensor units 220a, 220b. The wearing portion 210 in FIG. 17 is not limited to the illustrated shape and may have any shape that houses both the plurality of sensor units 220a, 220b and the below-described flexible substrate.

The plurality of sensor units 220a, 220b measure biological information on the subject while in contact with the subject's measured part. As illustrated in FIG. 17, in this embodiment, the plurality of sensor units include at least two sensors: the first sensor unit 220a and the second sensor unit 220b, which are biological sensors disposed according to a predetermined interval. As described below, these biological sensors include an optical emitter and an optical detector on a substrate. The interval ΔD between the first sensor unit 220a and the second sensor unit 220b is, for example, 10 mm to 30 mm. The first sensor unit 220a and the second sensor unit 220b acquire the pulse wave at different measured parts by an optical method. The pulse wave refers to a waveform representation, from the body surface, of the change in volume over time in a blood vessel due to inflow of blood. In other words, in this embodiment, the plurality of sensor units 220a, 220b optically acquire biological information.

The first sensor unit 220a for example includes two optical emitters 221a, 222a and an optical detector 223a. The second sensor unit 220b for example includes two optical emitters 221b, 222b and an optical detector 223b. The optical emitters 221a, 222a and 221b, 222b emit a measuring beam onto the measured part. This light passes through the body, light reaching the optical detectors 223a and 223b is detected, and the pulse waves are acquired. The optical emitters 221a, 222a and 221b, 222b include a light emitting element such as a Light Emitting Diode (LED) or a Laser Diode (LD). The optical detectors 223a and 223b include a light detecting element, such as a Photodiode (PD) or a Phototransistor (PT).

In the above-described configuration, each sensor unit is described as including two optical emitters and one optical detector. The sensor units in this embodiment, however, may perform measurement while including only one optical emitter and one optical detector. Nevertheless, as described above, a configuration including two optical emitters and one optical detector can improve the accuracy of measurement.

The optical emitters 221a, 222a and 221b, 222b for example emit green light (wavelength: 500 nm to 550 nm), red light (wavelength: 630 nm to 780 nm), or near infrared light (wavelength: 800 nm to 1600 nm). As compared to light of shorter wavelengths, light of longer wavelengths does not diminish until reaching a deeper position within the body. Therefore, by measuring biological information using an element that emits near infrared light, the measurement accuracy can be improved over the case of using an element that emits green light or red light.

In the wearing portion 210, the position of the openings 213a, 213b relative to the measured part is adjusted so that first sensor unit 220a and the second sensor unit 220b are both above the ulnar artery or the radial artery. The method for measuring the PWV at close proximity to the wrist using two acquired pulse waves is the same as in Embodiment 1, described with reference to FIG. 6. Details are therefore omitted.

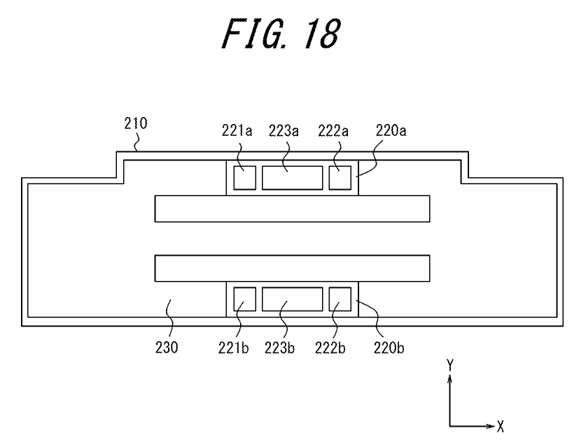

FIG. 18 is a top view illustrating the interior of the wearing portion 210 in FIG. 17. In other words, FIG. 18 illustrates the wearing portion 210 in FIG. 17 with the back face 211 removed.

As illustrated in FIG. 18, the first sensor unit 220a and the second sensor unit 220b are disposed on the flexible substrate 230 inside the wearing portion 210 constituting the housing of the measurement apparatus 200. FIG. 18 illustrates a configuration in which the back face 211 is separable from the wearing portion 210. The wearing portion 210 according to this embodiment, however, is not limited to such a configuration. The front face 212 may be separable, or an intermediate portion between the back face 211 and the front face 212 may be separable. Further, the wearing portion 210 according to this embodiment may have any configuration, such as an integral formation, that can house the plurality of sensor units 220a, 220b and the flexible substrate 230.

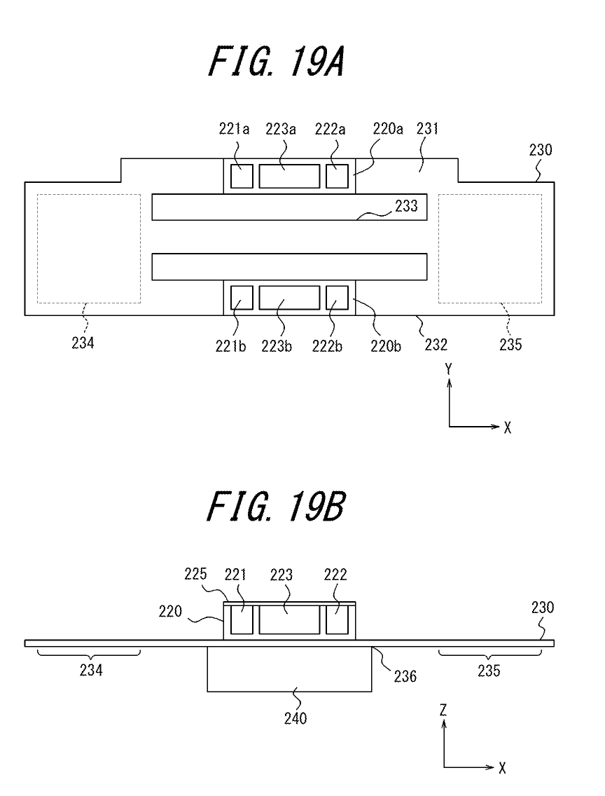

FIGS. 19A and 19B illustrate a state in which the flexible substrate 230 and other components in FIG. 18 are removed from the wearing portion 210. In other words, FIGS. 19A and 19B illustrate the flexible substrate 230 along with several other components. FIG. 19A is a view in the negative direction of the z-axis in FIG. 16, and FIG. 19B is a view in the positive direction of the Y-axis in FIG. 16.

As illustrated in FIG. 19A, the first sensor unit 220a is disposed in a first sensor installment area 231 of the flexible substrate 230. The second sensor unit 220b is disposed in a second sensor installment area 232 of the flexible substrate 230. The flexible substrate 230 includes a wiring passage 233 through which various wires can pass. At the back face of the flexible substrate 230, circuit installment areas 234, 235 can be provided for installment of circuits, such as the below-described controller. The circuit installment areas 234, 235 are indicated by dashed lines in FIG. 19A to indicate that these areas are provided at the back face of the flexible substrate 230.

In this manner, in this embodiment, two sensor units 220a, 220b are thus mounted on the flexible substrate 230, and the portion of the flexible substrate where these two sensor units 220a, 220b are mounted is divided into three or more parts. In other words, the two sensor units 220a, 220b in this embodiment are mounted on independent flexible substrates. Further, in this embodiment, the first sensor installment area 231 and second sensor installment area 232 on which these sensor units 220a, 220b are mounted are connected at both ends. Furthermore, the first sensor installment area 231 and second sensor installment area 232 on which the sensor units 220a, 220b are mounted and wired are also connected to both ends of the wiring passage 233 that is exclusively for one or more electrical wires.

As described above, since the sensor units 220a, 220b each include optical emitters and an optical detector, circuits for these elements are necessary. In order to reduce noise in such circuits, the circuits are preferably separated. In this embodiment, the wiring passage 233 that is a flexible substrate only for electrical wiring is provided separately from the first sensor installment area 231 and the second sensor installment area 232 of the flexible substrate 230. Therefore, by connecting both ends of the first sensor installment area 231 and the second sensor installment area 232 of the flexible substrate 230 with both ends of the wiring passage 233, electric circuits can be formed at both ends of each sensor unit. As a result, the circuits for the optical emitters and the circuits for the optical detectors can be disposed separately from the circuit installment areas 234, 235 in this embodiment, allowing a reduction in noise. As described above, starting with the controller, circuits for driving an optical semiconductor or for detection can be disposed in the circuit installment areas 234, 235. The electrical wiring connecting the circuit installment areas 234, 235 is formed in the wiring passage 233.

As illustrated in FIG. 19B, in the flexible substrate 230, an elastic body 240 is disposed in an elastic body installment area 236 provided on the opposite surface from the surface where the sensor units 220a, 220b are provided. As described below, this elastic body 240 can be configured using various elastic materials with an elasticity that exerts a restoring force on the sensor units 220 to an appropriate degree. From FIG. 19B onward, the sensor units 220a, 220b are at times abbreviated as “sensor units 220”. As illustrated in FIG. 19B, a protective surface 225 may be provided on the emitting surface of the optical emitters 221, 222 and the detecting surface of the optical detector 223, i.e. on the surface of the sensor units 220 that contacts the measured part. This protective surface 225 for example protects the optical emitters 221, 222 and the optical detector 223. The protective surface 225 may, for example, be a thin plate-shaped member that transmits light.

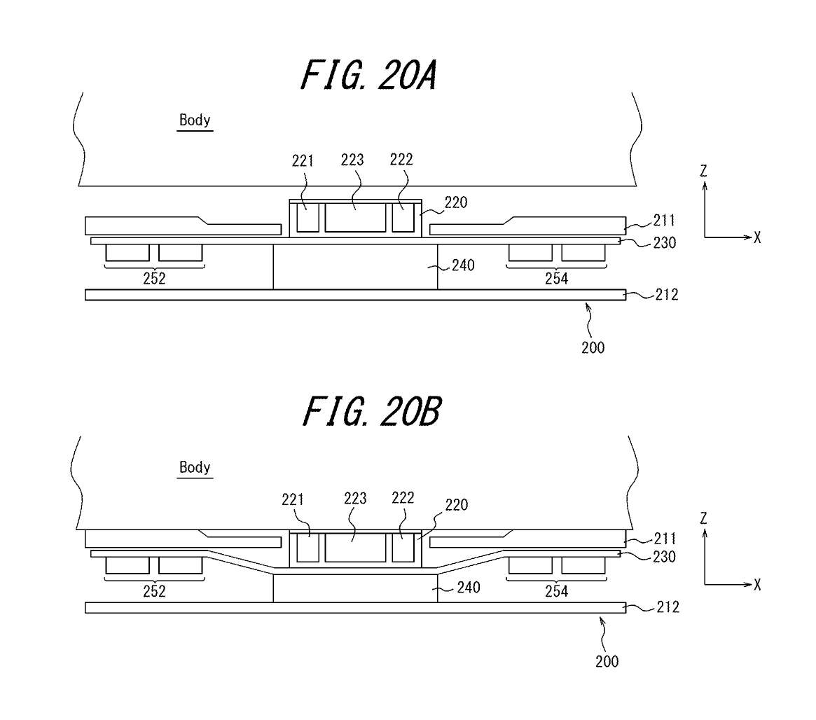

FIGS. 20A and 20B illustrate operations of the measurement apparatus 200 according to this embodiment. FIGS. 20A and 20B illustrate the measurement apparatus 200 as viewed from the same direction as in FIG. 19B.

FIG. 20A illustrates the state before measurement by the measurement apparatus 200, i.e. the state before the sensor units 220 contact the measured part of the living subject. In this state, since the measured part of the living subject is not in contact with the sensor units 220, the elastic body 240 pushes the sensor units 220 upward (in the positive direction of the z-axis) by virtue of the restoring force. Further, the elastic body 240 causes the sensor units 220 to project from the back face 211 of the wearing portion 210 through the opening 213 but does not cause the flexible substrate 230 on which the sensor units 220 are disposed to project through the opening 213. Hence, the state illustrated in FIG. 20A is maintained before the sensor units 220 contact the measured part of the living subject.

FIG. 20B illustrates the state at the start of and during measurement by the measurement apparatus 200, i.e. the state when the sensor units 220 are contacted to the measured part of the living subject and are further pushed. In this state, the measured part of the living subject is in contact with the sensor units 220 and is further pushed. The elastic body 240 then undergoes elastic deformation because of the pressing force of the measured part, and the sensor units 220 are pushed downward (in the negative direction of the z-axis). In this embodiment, the sensor units 220 are disposed on the flexible substrate 230. Therefore, upon the sensor units 220 being pushed downward, the flexible substrate 230 deforms (bends) from the pressing force. Also, in this case as well, the elastic body 240 pushes upward (in the positive direction of the z-axis) on the sensor units 220 by the restoring force. Accordingly, in the state in which the sensor units 220 contact and are pressed by the measured part of the living subject, the sensor units 220 are in close contact with the subject's measured part as a result of an appropriate pressing force. This close contact improves the measurement accuracy of biological information.

As illustrated in FIG. 20B, if the measured part of the living subject pushes the sensor units 220, the subject's measured part is pressed and may sink slightly. In such a case, depending on the part of the body, the surface of the body may contact the back face 211 of the wearing portion 210. The pressure state is maintained in this case as well, however, since the elastic body 240 pushes upward (in the positive direction of the z-axis) on the sensor units 220. Furthermore, as illustrated in FIGS. 20A and 20B, contact between the back face 211 of the wearing portion 210 and the surface of the body can be avoided near the sensor units 220 by making the back face 211 relatively thin near the sensor units 220 or making the back face 211 relatively thick at locations not near the sensor units 220. Accordingly, in this embodiment, the sensor units 220 can be brought into close contact with the subject's measured part reliably at a predetermined pressing force or less.

After measurement by the measurement apparatus 200 is complete in the state illustrated in FIG. 20B, when the measured part of the living subject is removed from the sensor units 220, the measurement apparatus 200 then returns to the state illustrated in FIG. 20A as a result of the elastic force of the elastic body 240. FIGS. 20A and 20B illustrate an example in which circuits 252 for the optical emitters are disposed in the circuit installment area 234 illustrated in FIGS. 19A and 19B. Similarly, FIGS. 20A and 20B illustrate an example in which circuits 254 for the optical detectors are disposed in the circuit installment area 235 illustrated in FIGS. 19A and 19B.

Furthermore, in this embodiment, the flexible substrate 230 is connected only at both ends to the first sensor installment area 231 where the first sensor 220a is installed and the second sensor installment area 232 where the second sensor 220b is installed, as illustrated in FIG. 19A. Therefore, the first sensor 220a and the second sensor 220b can move independently on the flexible substrate 230. Thus by virtue of a structure which partially separates the flexible substrate 230, the two sensor units can separately contact an uneven surface on the measured part of the living subject. For example, if two sensors are disposed on a single, non-flexible member and placed in close contact with a human body, it is assumed that pushing one of the sensors affects the other sensor. In this embodiment, however, use of the partially separated flexible substrate 230 allows two sensors that are brought into close contact with a human body to be pushed individually while each conforms to an uneven surface of the human body.

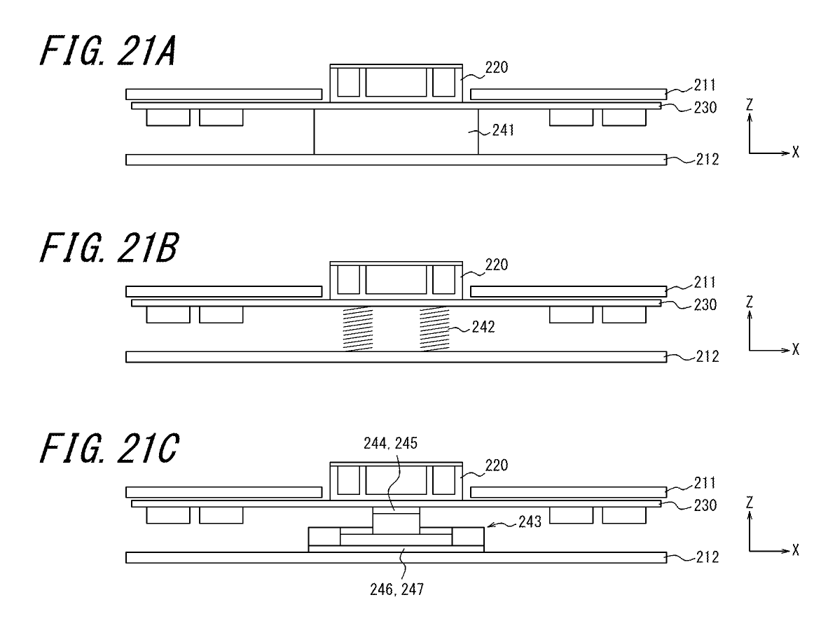

FIGS. 21A, 21B, and 21C illustrate concrete examples of the elastic body 240.

As described above, in this embodiment, the elastic body 240 is provided on the opposite surface of the flexible substrate 230 from the surface where the sensor units are provided. Various elastic members configured to push back on the sensor units 220 with a restoring force corresponding to the pressing force when the sensor units 220 are pressed may be used in the elastic body 240. Accordingly, for example a member such as the sponge or urethane 241 in FIG. 21A, the coil spring 242 in FIG. 21B, or the plate spring 243 in FIG. 21C may be used. In FIGS. 21A, 21B, and 21C, a structure such as the one in FIG. 20 is not illustrated, i.e. a structure in which the back face 211 near the sensor units 220 is relatively thin, or the back face 211 at locations not near the sensor units 220 is relatively thick.

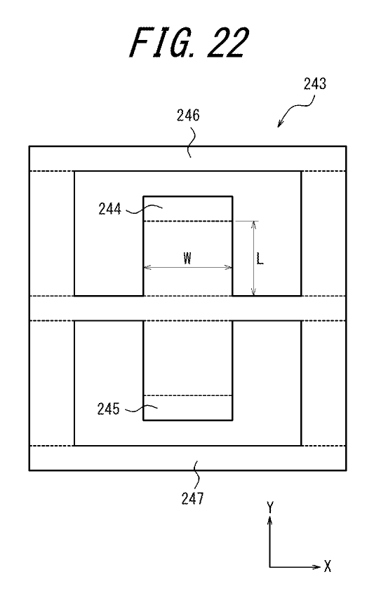

The plate spring 243 in FIG. 21C may, for example, be a member with the shape illustrated in FIG. 22 when viewed in the z-axis direction. As illustrated in FIG. 21C and FIG. 22, outer spring portions 246, 247 of the plate spring 243 are in contact with the front face 212 of the wearing portion 210 and exert an elastic force in the negative direction of the z-axis. Furthermore, an inner spring portion 244 of the plate spring 243 is in contact with the first sensor installment area 231 of the flexible substrate 230 and pushes the first sensor unit 220a in the positive direction of the z-axis. Similarly, an inner spring portion 245 of the plate spring 243 is in contact with the second sensor installment area 232 of the flexible substrate 230 and pushes the second sensor unit 220b in the positive direction of the z-axis. An elastic force is exerted on the front face 212 of the wearing portion 210 in the negative direction of the z-axis.

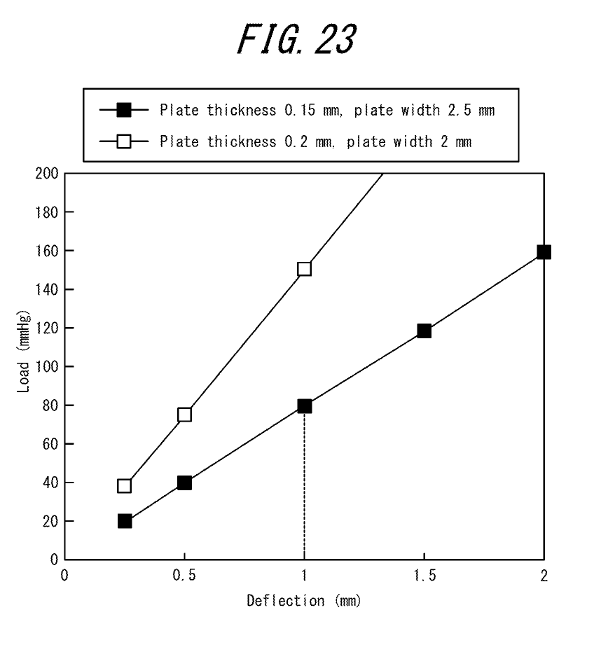

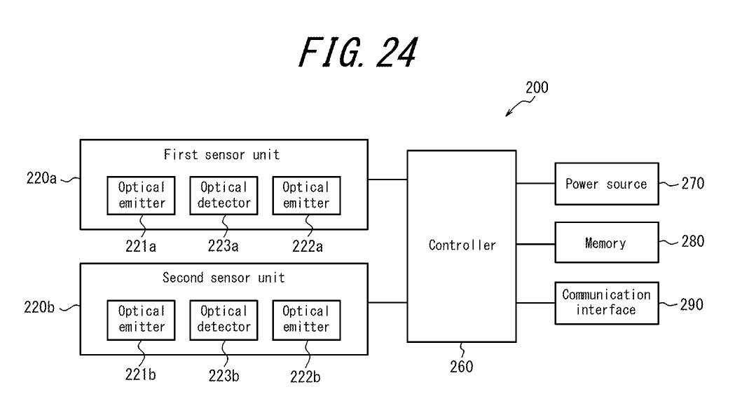

FIG. 23 is a graph of the relationship between deflection (mm) and load (mmHg) for the plate spring with the shape in FIG. 22. As examples, FIG. 23 illustrates the case of a plate thickness (thickness in the z-axis direction in FIG. 22) of 0.15 mm and a plate width (width W in FIG. 22) of 2.5 mm and the case of a plate thickness of 0.2 mm and a plate width of 2 mm. In both cases, the effective operating length of the spring (the length L in FIG. 22) is 7.5 mm.

In this way, the plurality of sensor units 220a, 220b are supported by the wearing portion 210 and acquire the subject's biological information while in contact with the subject's measured part in this embodiment. The plurality of sensor units 220a, 220b in this embodiment are displaceably supported relative to the wearing portion 210.

The plurality of sensor units 220a, 220b are supported by the wearing portion 210 via the elastic body 240. The elastic body 240 may, for example, be a member such as a spring. In this way, in this embodiment, at least one of the plurality of sensor units 220a, 220b is displaceably supported relative to the wearing portion 210 via the elastic body 240 while the wearing portion 210 is worn by the subject. At least one of the plurality of sensor units 220a, 220b is displaceable with respect to the wearing portion 210 in the direction of the front face 212 and the back face 211 (in the negative direction of the z-axis) of the wearing portion 210. At least one of the plurality of sensor units 220a, 220b is displaceable with respect to the wearing portion 210 in a plane parallel to the extending direction of the wearing portion 210. Furthermore, at least one of the plurality of sensor units 220a, 220b is also displaceable so as to tilt relative to the front face 212 and the back face 211 of the wearing portion 210.

The plurality of sensor units 220a, 220b at least include sensors that detect the subject's biological information and a substrate on which the sensors are mounted. The sensors typically may include the optical detectors 223a, 223b. At least a portion of the substrate is preferably flexible, and a typical example of the substrate is the flexible substrate 230. In this case, the plurality of sensor units 220a, 220b can be configured to include the optical detectors 223a, 223b and at least the portions of the flexible substrate (for example, 231, 232) on which these sensors are mounted. The sensor installment areas 231 and 232 need not be separated if the flexible substrate is flexible enough for the sensor units 220a, 220b each to come into close contact with the subject.

Next, the measurement apparatus 200 is described from a functional perspective.

FIG. 24 is a functional block diagram schematically illustrating the structure of the measurement apparatus 200 in FIG. 16. The measurement apparatus 200 includes the first sensor unit 220a, the second sensor unit 220b, a controller 260, a power source 270, a memory 280, and a communication interface 290. In this embodiment, the first sensor unit 220a, second sensor unit 220b, controller 260, power source 270, memory 280, and communication interface 290 may each be included inside the wearing portion 210.

The first sensor unit 220a and the second sensor unit 220b each include a biological sensor, as described above, and acquire biological information from the measured part. The first sensor unit 220a includes the optical emitters 221a, 222a and the optical detector 223a. The second sensor unit 220b includes the optical emitters 221b, 222b and the optical detector 223b.

The controller 260 is a processor that, starting with the functional blocks of the measurement apparatus 200, controls and manages the measurement apparatus 200 overall. Further, the controller 260 is also a processor that calculates the PWV using the pulse waves acquired as biological information. The controller 260 can be configured in a similar way to the controller 143 described in Embodiment 1.

The power source 270 can be configured in a similar way to the power source 144 described in Embodiment 1.

The memory 280 can be configured in a similar way to the memory 145 described in Embodiment 1.

The communication interface 290 can be configured in a similar way to the communication interface 146 described in Embodiment 1.

Next, the method for using the measurement apparatus 200 is described. As was described with reference to FIG. 16, the subject performs measurement with the measurement apparatus 200 by wrapping the measurement apparatus 200 around the wrist. When wrapping the measurement apparatus 200 around the wrist, the subject wraps the wearing portion 210 (or the bands 214, 215 of the wearing portion 210) around the wrist after adjusting the position of the sensor units 220 so that a measurement beam is emitted from the optical emitters of the sensor units 220 onto the ulnar artery or the radial artery for which biological information is to be acquired.

As illustrated in FIGS. 20A and 20B, the measurement apparatus 200 is worn by the subject with the two sensor units 220 in contact with a measured part such as the wrist. The sensor units 220 preferably are made to contact the wrist at positions where the measurement beam is emitted onto the ulnar artery or the radial artery, by adjustment at the time the subject wears the apparatus.

While the measurement apparatus 200 is worn, the two sensor units 220 are in close contact with the subject's wrist by virtue of the elastic force of the elastic body 240. By the sensor units 220 being in close contact with the wrist, the positional relationship between the wrist and the sensor units 220 tends not to change, allowing improvement in the measurement accuracy of the sensor units 220.

Here, the two sensor units 220 are independently displaceably supported relative to the wearing portion 210. Therefore, each of the two sensor units 220 more easily comes in close contact with the wrist, which is the measured part. Also, if the wearing portion 210 shifts relative to the wrist, the sensor units 220 are each displaced, making it easier to maintain close contact between the sensor units 220 and the wrist. Therefore, the positional relationship between the measurement unit 220 and the wrist tends to remain unchanged, and the conditions for measurement of biological information by the measurement unit 220 do not change easily. Furthermore, even if pressure is not applied evenly in the same direction to the plurality of sensor units 220, the plurality of sensor units 220 are still each in close contact with the subject's measured part because of an appropriate pressing force. With this configuration, the measurement apparatus 200 can improve the measurement accuracy of biological information by the sensor units 220.

The sensor units 220 are configured to be in contact with the wrist at a predetermined pressure or less while the measurement apparatus 200 is worn. During measurement of biological information, the sensor units 220 may always contact the wrist at a predetermined pressure or less, regardless of movement by the subject. The predetermined pressure is determined on the basis of factors such as the biological information measured by the measurement apparatus 200 and the configuration of the measurement apparatus 200. The predetermined pressure is preferably a pressure at which error tends not to occur in the measurement results of the biological information. In this embodiment, the measurement apparatus 200 measures the PWV as the biological information. The predetermined pressure is therefore preferably a pressure at which error tends not to occur in the measurement results of the PWV.

Since the preferred values for the predetermined pressure in the measurement apparatus 200 according to this embodiment are similar to those described in Embodiment 1 with reference to FIG. 11, a detailed description is omitted here. The measurement apparatus 200 is configured so that when worn, the sensor units 220 contact the measured part at a pressure of 80 mmHg or less. An elastic body that can achieve this pressure is used in the measurement apparatus 200 as the elastic body 240.

In this way, in this embodiment, the plurality of sensor units 220a, 220b are preferably arranged along a predetermined blood vessel of the subject when the measurement apparatus 200 is worn on the subject's wrist. Also, in this embodiment, at least one of the plurality of sensor units 220a, 220b is in contact with the measured part at a predetermined pressure or less when the wearing portion 210 is worn by the subject.

In this embodiment, the plurality of sensor units 220a, 220b are preferably arranged so as to contact measured parts that are a predetermined distance apart in the direction of a predetermined blood vessel of the subject. In other words, as illustrated in FIG. 17, with the direction of ΔD as the direction of the predetermined blood vessel of the subject, the plurality of sensor units 220a and 220b are preferably arranged to contact measured parts that are separated by the predetermined distance of ΔD.

Furthermore, in this embodiment, among the measured parts contacted by the plurality of sensor units 220a, 220b, the sensor unit in contact with the measured part closer to the subject's heart along the predetermined blood vessel is preferably configured to contact the measured part at a predetermined pressure or less. In other words, when the subject wraps the wearing portion 210 of the measurement apparatus 200 around the wrist, the positive direction of the y-axis in FIG. 17 is in the direction of the subject's upper arm, and the negative direction of the y-axis is in the direction of the subject's palm. In this case, the positive direction of the y-axis, i.e. the direction of the subject's upper arm, is closer to the subject's heart along the predetermined blood vessel. Accordingly, in this case, at least the first sensor unit 220a is configured to contact the measured part at a predetermined pressure or less.

With the measurement apparatus 200 according to this embodiment, because the sensor units 220 that measure the biological information contact the measured part at a predetermined pressure or less, the measurement accuracy of biological information can therefore be improved by the setting of the predetermined pressure. For example, in the above embodiment, the measurement accuracy of the PWV can be improved by setting the predetermined pressure to 80 mmHg.

In the measurement apparatus 200 according to this embodiment, the plurality of sensor units 220 are independently displaceably supported relative to the wearing portion 210. Therefore, with this measurement apparatus 200, even if the wearing portion 210 shifts during measurement of biological information, by virtue of the positional relationship between the sensor units 220 and the wearing portion 210 changing, a change in the degree of close contact between the sensor units 220 and the measured part is prevented. The measurement conditions therefore do not change easily with respect to the position of the sensor unit 220 relative to the measured part during measurement of biological information with the measurement apparatus 200, allowing improvement in the measurement accuracy of biological information. Also, during measurement of biological information, the sensor units 220 can always contact the wrist at a predetermined pressure or less, regardless of movement by the subject.

This disclosure is not limited to the above embodiments, and a variety of modifications and changes are possible. For example, the functions and the like included in the various components may be reordered in any logically consistent way. Furthermore, components may be combined into one or divided.

For example, in the above embodiment, the measurement apparatus 200 was described as including two sensor units: the first sensor unit 220a and the second sensor unit 220b. The plurality of sensor units in this disclosure are not limited in number to two sensor units, however, and may be any number two or greater. In this case, the shape of the flexible substrate 230 is preferably modified in accordance with the number of sensor units.

In the above embodiments, the substrate on which sensors such as optical emitters and optical detectors are mounted was described as being the flexible substrate 230, but in this disclosure it is not necessary for the entire substrate to be flexible. Since it suffices for the plurality of sensor units to be independently moveable, it is enough for at least a portion of the substrate to be flexible.

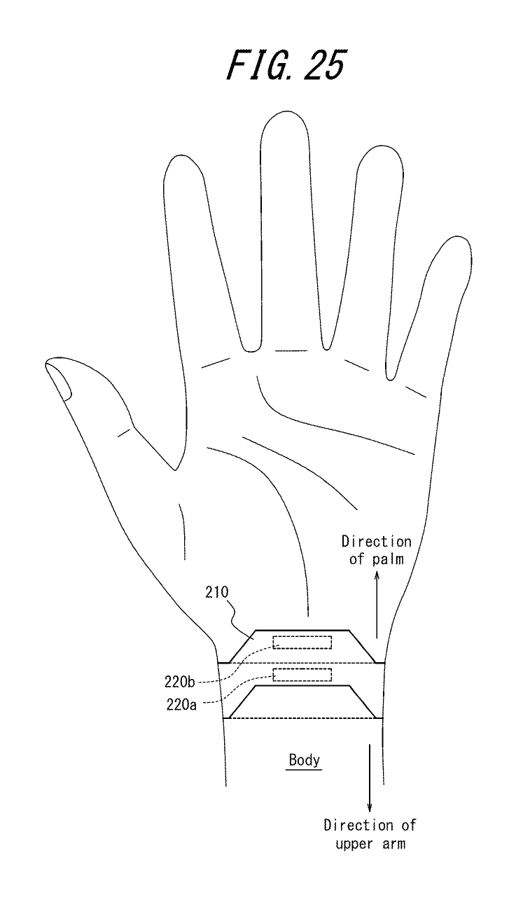

In the above embodiment, the wearing portion 210 need not have the shape illustrated in FIG. 16. For example when the measurement apparatus 200 is worn on the subject's wrist, at least a portion of the wearing portion 210 may be offset in the direction of the subject's upper arm. In the example in FIG. 25, when the measurement apparatus 200 is worn, the location of the sensor units 220 in the wearing portion 210 is above the wrist. The remainder of the wearing portion 210 is offset from the location of the sensor units 220 in the direction of the upper arm. As a result, the sensor units 220 are in contact with the measured part of the wrist, whereas the remainder of the wearing portion 210 is shifted towards the upper arm from the wrist. Movement of the subject's wrist is therefore less impeded. In other words, this configuration thus reduces interference by the wearing portion 210 with the range of motion of the subject's wrist.

<Sensor System 1>

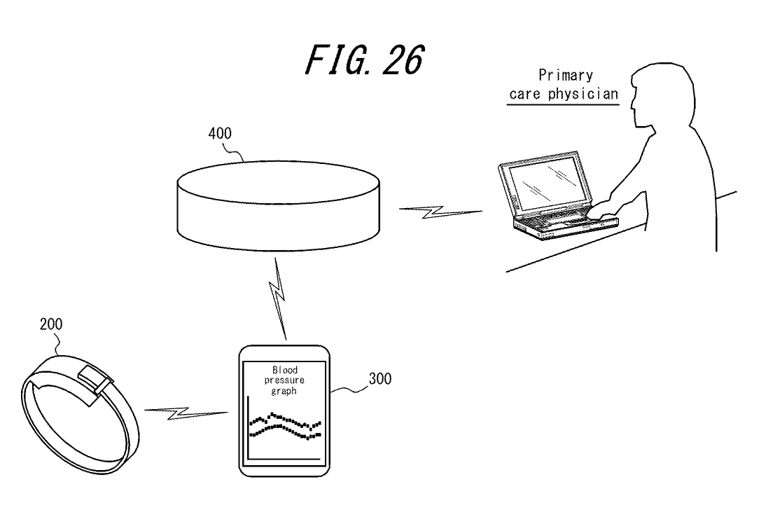

With reference to FIG. 26, the following describes a sensor system 1 including the above-described measurement apparatus 200.

In addition to the measurement apparatus 200, the sensor system 1 includes a display apparatus 300 and a server 400. The display apparatus 300 collects sensor signals acquired by the measurement apparatus 200 and performs a variety of information processing. Collection of the sensor signals is performed by the measurement apparatus 200 transmitting data to the display apparatus 300 over a wired or wireless communication network. On a display, the display apparatus 300 displays biological information based on the sensor signals acquired by the measurement apparatus 200. The display apparatus 300 also displays information subjected to information processing by the server 400 on the display. The display apparatus 300 may be configured as a dedicated terminal including a display such as an LCD or may be configured as a general terminal such as a smartphone or a tablet PC.

The server 400 collects biological information on the subject from the display apparatus 300 and performs a variety of information processing. Collection of the biological information is performed by the display apparatus 300 transmitting data to the server 400 over a wired or wireless communication network. The server 400 also transmits the results of information processing performed using the biological information to the display apparatus 300. An existing server including a controller with a memory and a CPU may, for example, be used as the server 400. The memory may be a semiconductor memory or other memory.

More concretely, in the sensor system 1, sensor signals acquired by the measurement apparatus 200 are transmitted by the communication interface of the measurement apparatus 200 to the display apparatus 300. Furthermore, the biological information acquired by information processing performed on the sensor signals in the display apparatus 300 is transmitted to the server 400 by a communication interface of the display apparatus 300. Once the server 400 receives the biological information transmitted by the display apparatus 300, the controller of the server 400 performs various information processing in accordance with the received biological information of the subject. For example, the server 400 can store the biological information transmitted from the display apparatus 300 in the memory of the server 400 as time-series data along with the acquisition time of the sensor signals. The controller of the server 400 for example compares the stored data with past data of the same subject or data of another subject already stored in the memory of the server 400. The controller then generates the best advice for the subject using the result of comparison. The communication interface of the server 400 transmits the acquired time-series data of the subject and the generated advice to the display apparatus 300. The display apparatus 300 displays the received data and advice on the screen. As necessary, the server 400 may also for example transmit the time-series data of the subject to the subject's primary care physician. Further, the server 400 may also, for example, transmit the advice to the subject's primary care physician as necessary. Further, a functional component with the same functions as the memory and controller of the server 400 may also be provided in the measurement apparatus 200 or the display apparatus 300. In this case, the sensor system 1 need not include the server 400.

The measurement apparatuses 100, 200 may include a notification interface that notifies the subject of the result of measuring biological information. The notification unit can provide notification with any method recognizable by the subject. The notification unit can, for example, provide notification by sound, image, vibration, or a combination thereof. The method of providing notification with the notification unit is not limited to these examples.

In the above embodiments, the measurement apparatuses 100, 200 are described as being used while wrapped around the subject's wrist, but the mode of use of the measurement apparatuses 100, 200 is not limited to this case. In accordance with the position of the measured part, the measurement apparatuses 100, 200 may, for example, be used while worn on a part of the body other than the wrist, such as the ankle.

In the above embodiments, the measurement apparatuses 100, 200 are examples of apparatuses for measuring PWV, but this disclosure is not limited to this case. The measurement apparatuses 100, 200 can very accurately acquire the pulse wave and may therefore be apparatuses that measure biological information using the pulse wave. The measurement apparatuses 100, 200 may, for example, measure blood pressure or pulse from the acquired pulse wave.

Much of the subject matter of the present disclosure is described as a series of operations executed by a computer system and other hardware that can execute program instructions. Examples of the computer system and other hardware include a general-purpose computer, a Personal Computer (PC), a dedicated computer, a workstation, a Personal Communications System (PCS), an electronic notepad, a laptop computer, and other programmable data processing apparatuses. It should be noted that in each embodiment, various operations are executed by a dedicated circuit (for example, individual logical gates interconnected in order to execute a particular function) implemented by program instructions (software), or by a logical block, program module, or the like executed by one or more processors. The one or more processors that execute a logical block, program module, or the like are, for example, one or more of each of the following: a microprocessor, a central processing unit (CPU), an Application Specific Integrated Circuit (ASIC), a Digital Signal Processor (DSP), a Programmable Logic Device (PLD), a Field Programmable Gate Array (FPGA), a controller, a microcontroller, an electronic device, another apparatus designed to be capable of executing the functions disclosed herein, and/or a combination of any of the above. The embodiments disclosed herein are, for example, implemented by hardware, software, firmware, middleware, microcode, or a combination of any of these.

The machine-readable, non-transitory storage medium used here may also be configured by a computer-readable, tangible carrier (medium) in the categories of solid-state memory, magnetic disks, and optical discs. Data structures and an appropriate set of computer instructions, such as program modules, for causing a processor to execute the techniques disclosed herein are stored on these media. Examples of computer-readable media include an electrical connection with one or more wires, a magnetic disk storage medium, or another magnetic or optical storage medium (such as a Compact Disc (CD), Digital Versatile Disc (DVD®), and Blu-ray Disc® (DVD and Blu-ray disc are each a registered trademark in Japan, other countries, or both)), portable computer disk, Random Access Memory (RAM), Read-Only Memory (ROM), rewritable programmable ROM such as EPROM, EEPROM, or flash memory, another tangible storage medium that can store information, or a combination of any of these. The memory may be provided internal and/or external to a processor/processing unit. As used in the present disclosure, the term “memory” refers to all types of long-term storage, short-term storage, volatile, non-volatile, or other memory. No limitation is placed on the particular type or number of memories, or on the type of medium for memory storage.

According to embodiments of this disclosure, a measurement apparatus that can improve the measurement accuracy of biological information and a sensor system that includes a measurement apparatus that measures biological information can be provided.

1. A measurement apparatus comprising:
a wearing portion to be worn by a subject; and
at least one sensor supported by the wearing portion and configured to acquire biological information of the subject while in contact with a measured part of the subject, wherein
the sensor contacts the measured part at a predetermined pressure or less while the wearing portion is worn by the subject.

2. The measurement apparatus of claim 1, wherein the sensor is displaceably supported relative to the wearing portion.

3. The measurement apparatus of claim 1, wherein
the at least one sensor comprises a plurality of sensors, and
one or more sensors among the plurality of sensors contact the measured part at a predetermined pressure or less while the wearing portion is worn by the subject.

4. The measurement apparatus of claim 3, wherein
the plurality of sensors contact measured parts that are a predetermined distance apart in a direction of a predetermined blood vessel of the subject, and
among the measured parts contacted by the plurality of sensors, a measured part located closer to the heart of the subject along the predetermined blood vessel is contacted by one of the plurality of sensors at the predetermined pressure or less.

5. The measurement apparatus of claim 3, wherein each of the plurality of sensors is displaceably supported relative to the wearing portion.

6. The measurement apparatus of claim 3, wherein each of the plurality of sensors is arranged along a predetermined blood vessel of the subject while the measurement apparatus is worn on a wrist of the subject.

7. The measurement apparatus of claim 3, wherein the plurality of sensors include at least a sensor configured to detect the biological information of the subject and a substrate, the sensor being mounted on the substrate, and at least a portion of the substrate being flexible.

8. The measurement apparatus of claim 1, wherein the sensor is supported relative to the wearing portion by an elastic body.

9. The measurement apparatus of claim 8, wherein the elastic body is a spring.

10. The measurement apparatus of claim 1, wherein the wearing portion is a band worn on a wrist of the subject.

11. The measurement apparatus of claim 10, wherein a position of at least a portion of the band is shifted, relative to the measured part contacted by the sensor, in a direction of an upper arm of the subject while the measurement apparatus is worn on the wrist.

12. The measurement apparatus of claim 1, wherein the predetermined pressure is an average blood pressure of the subject.

13. The measurement apparatus of claim 1, wherein the predetermined pressure is 80 mmHg.

14. The measurement apparatus of claim 1, wherein the biological information is a pulse wave.

15. The measurement apparatus of claim 14, further comprising a controller configured to calculate a pulse wave velocity using the pulse wave.

16. The measurement apparatus of claim 1, wherein the sensor optically acquires the biological information.

17. The measurement apparatus of claim 1, wherein
the wearing portion comprises a plurality of supports, and
the plurality of supports and the sensor are in close contact with the subject while the wearing portion is worn by the subject.

18. A measurement apparatus comprising:
a wearing portion to be worn by a subject; and
at least one sensor supported by the wearing portion and configured to acquire biological information of the subject while in contact with a measured part of the subject, wherein
the sensor is supported by an elastic body to be displaceable relative to the wearing portion while the wearing portion is worn by the subject.

19. The measurement apparatus of claim 18, wherein
the at least one sensor comprises a plurality of sensors, and
one or more sensors among the plurality of sensors are supported by the elastic body to be displaceable relative to the wearing portion while the wearing portion is worn by the subject.

20. A sensor system comprising:
a measurement apparatus comprising a wearing portion to be worn by a subject and a sensor supported by the wearing portion and configured to acquire biological information of the subject while in contact with a measured part of the subject, the sensor contacting the measured part at a predetermined pressure or less while the wearing portion is worn by the subject; and
a display apparatus configured to display biological information by referring to a sensor signal acquired by the sensor.
